(19) United States
(12) Patent Application Publication
(10) Pub. No.: US 2002/0046471 A1
(43) Pub. Date: Apr. 25, 2002

(54) Methods and device for estimating body weight or ease of parturition of a ruminant

(76) Inventor: Andrew L. Skidmore, Alexander, NY (US)

Correspondence Address: Georgia Evans, Esq., NIXON PEABODY LLP, Clinton Square, P.O. Box 31051, Rochester, NY, 14603, US

(21) Appl. No.: 09/886,761

(22) Filed: Jun. 21, 2001

Related U.S. Application Data
(60) Provisional application No. 60/213,332, filed on Jun. 22, 2000.

(51) Int. Cl. G01B 5/02

(52) U.S. Cl. 33/511; 33/494

(57) ABSTRACT

A method for estimating a body weight of a ruminant with a skeletal structure including a cranial portion, a caudal portion, and hip joints located between the cranial and caudal portions including measuring a distance between hip joints of a ruminant and converting the measured distance to an estimated body weight of the ruminant.

The present invention claims the benefit of U.S. Provisional Patent Application Ser. No. 60/213,332, filed Jun. 22, 2000, which is hereby incorporated by reference in its entirety.

### FIELD OF THE INVENTION

The present invention relates to methods for estimating a body weight of a ruminant, methods for estimating ease of parturition of a ruminant, and an apparatus for estimating a body weight or ease of parturition of a ruminant.

### BACKGROUND OF THE INVENTION

Those raising dairy heifers strive to raise heifers of an adequate size to produce calves at between about 22 and 24 months, with acceptable levels of milk production. Calving age is highly correlated to the profitability of the animal, and body weight after calving is highly correlated to first lactation milk production. It is necessary to provide proper nutrition and feeding management so that heifers are large enough to breed at 13 to 15 months old in order to reach these goals.

Accordingly, it is desirable for those raising dairy heifers to be able to accurately evaluate their heifer-rearing program by monitoring the height and weight of calves and heifers and comparing the results against breed averages for a specific age group. However, it is difficult to visually determine whether a heifer or calf is of normal height and/or weight for her age. In addition, few diary raisers have complete restraint and handling systems or animal scales to determine body weights. Therefore, techniques for estimating body weight from other body measurements are needed.

Several techniques have been developed to estimate a diary animal's body weight, including heart girth measurements, withers height measurements, hip height measurements, body length measurements, and hip width measurements. Heart girth measurements are made by pulling a weight tape around the heart girth just behind the front legs and shoulder blades and are accurate to within 5-7% of the actual body weight (see, e.g., Heinrichs et al., Monitoring Dairy Heifer Growth, Pennsylvania State University (1998)). Withers height measurements and hip height measurements are made using a measuring stick to measure the animal at the highest point of the withers and the highest point of the hips, respectively (see, e.g., Heinrichs et al., Monitoring Dairy Heifer Growth, Pennsylvania State University (1998)). Body length measurements are made by placing a measuring tape along the length of the animal's body, with the animal straight and with its head held forward and upright (see, e.g., Heinrichs et al., Monitoring Dairy Heifer Growth, Pennsylvania State University (1998)). Hip width measurements are made using calipers which are placed against the widest part of the hip bone—the tuber coxi (see, e.g., Heinrichs et al., Monitoring Dairy Heifer Growth, Pennsylvania State University (1998)). However, in all of the above techniques, significant animal restraint and handling is needed to measure the animal, particularly when making measurements at positions that are difficult to reach, such as the tuber coxi. In addition, for heart girth measurements, withers height measurements, and body length measurements, the animal must be restrained in a particular position. Further, in all of the above techniques, those measuring the animal must be in close proximity to the animal, thus increasing the risk of injury. Moreover, in the above techniques the devices used for making the desired measurements are difficult to operate, leading to decreased accuracy in the obtained measurements.

In addition, it is desirable for those raising dairy heifers to be able to accurately estimate the ease of parturition or calving for the heifers. Such estimates are typically made by measuring the internal area of the birth canal. However, as above, significant animal restraint and handling is needed to make these measurements.

### SUMMARY OF THE INVENTION

The present invention relates to a method for estimating a body weight of a ruminant with a skeletal structure including a cranial portion, a caudal portion, and hip joints located between the cranial and caudal portions. The method includes measuring a distance between hip joints of a ruminant and converting the measured distance to an estimated body weight of the ruminant.

The present invention also relates to a method for estimating ease of parturition of a ruminant with a skeletal structure including a cranial portion, a caudal portion, and hip joints located between the cranial and caudal portions. This method includes measuring a distance between hip joints of a ruminant and converting the measured distance to an estimated ease of parturition value of the ruminant.

Another aspect of the present invention relates to a method for estimating a body weight or ease of parturition of a ruminant with a skeletal structure including a cranial portion having first and second sides, a caudal portion having first and second sides, and hip joints located between the cranial and caudal portions. This method includes measuring a distance behind tuber coxi of a ruminant and between first and second sides of a caudal portion of the ruminant and converting the measured distance to an estimated body weight or ease of parturition of the ruminant.

Yet another aspect of the present invention relates to an apparatus for estimating a body weight or ease of parturition of a ruminant. The apparatus includes a first arm having proximal and distal ends, a second arm having proximal and distal ends, wherein the first and second arms are pivotally mounted to each other between the proximal and distal ends of the first and second arms, and a scale device connected to one of the first and second arms between the proximal and distal ends and overlapping the other one of the first arm and the second arm between the proximal and distal ends.

The apparatus of the present invention allows the operator to stand farther away from the animal being measured and limited animal restraint or handling is needed to operate the device, thus improving operator safety. In addition, the apparatus is faster and easier to operate than prior art devices, again improving operator safety. The method and apparatus of the present invention can be used to estimate ease of parturition or calving. In addition, the apparatus of the present invention allows more accurate measurement of body weight and ease of parturition, because the improved ease of use of the apparatus results in more accurate measurements of pelvic widths, thus leading to more accurate estimates of body weight and ease of parturition.

### BRIEF DESCRIPTION OF THE DRAWINGS

FIG. 1 is a top view of an apparatus for estimating body weight or ease of parturition in accordance with one embodiment of the present invention;

FIG. 2 is a top view of an apparatus for estimating body weight or ease of parturition in accordance with a second embodiment of the present invention;

FIG. 3 is a side view of the apparatus of FIG. 2;

FIG. 4 is a top view of an apparatus for estimating body weight or ease of parturition in accordance with a third embodiment of the present invention;

FIG. 5 is a top view of an apparatus for estimating body weight or ease of parturition in accordance with a fourth embodiment of the present invention;

FIGS. 6A and B show the use of an apparatus for estimating body weight or ease of parturition in accordance with the present invention;

FIG. 7 is a leverage plot for the effect of pelvic width of Holstein heifers;

FIG. 8 is a whole-model leverage plot of the observed weight (kg) of Holstein heifers versus their predicted weight (kg) by pelvic width; and

FIG. 9 is a graph showing the relationship of pelvic width (cm) to scale weight (kg) for Holstein heifers.

### DETAILED DESCRIPTION OF THE INVENTION

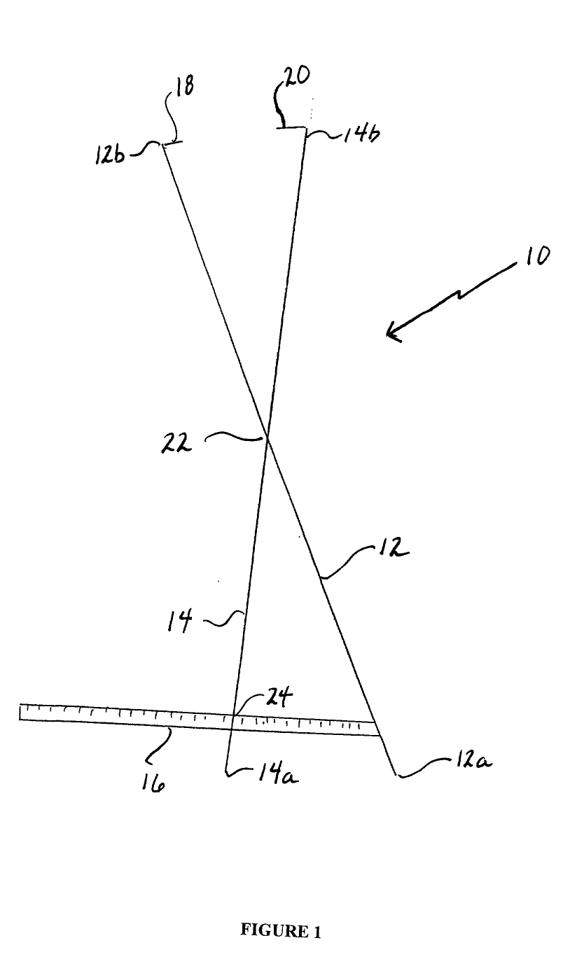

An apparatus 10 for estimating body weight or ease of parturition of a ruminant in accordance with one embodiment of the present invention is shown in FIG. 1. The apparatus includes a first arm 12, a second arm 14, and a scale device 16. Advantages of the apparatus 10 of the present invention include improved operator safety and ease in manufacturing and use. In addition, the apparatus of the present invention allows more accurate measurement of body weight and ease of parturition.

Referring to FIG. 1, the apparatus 10 includes a first arm 12. The first arm 12 has a proximal end 12a and a distal end 12b. In this particular embodiment, first arm 12 is substantially straight. The first arm 12 is typically rectangular in cross section, however, other shapes, such as elongated cylinders, can be used. Further, the first arm 12 is typically about 0.75 to about 1 inch in thickness (or diameter), although other dimensions may be used. The first arm 12 is typically from about 35 inches to about 40 inches in length, which allows the user to maintain a safe distance from a ruminant when using the apparatus 10 of the present invention. However, the first arm 12 may be of any suitable length, based on the size of the ruminant. In particular, the larger the ruminant to be measured, the longer the arm length.

In the embodiment shown in FIG. 1, the first arm 12 includes at its distal end 12b a contacting portion 18 extending substantially perpendicularly from the first arm 12. The contacting portion 18 makes contact with the ruminant for which an estimate of body weight or ease of parturition is desired. The contacting portion 18 may be integrated with or attached to the first arm 12. Suitable attachment mechanisms include, for example, rivets, bolts, and adhesives.

Also, in this particular embodiment, the first arm 12 is made of a rigid material. Use of a rigid material allows the apparatus to be extremely sturdy which, in turn, produces more accurate measurements. In this particular embodiment, the first arm 12 is made of hardwood, such as maple or oak. Other suitable rigid materials include other woods, metals, and hard plastics.

The apparatus 10 of FIG. 1 also includes a second arm 14. The second arm 14 includes a proximal end 14a and a distal end 14b. The second arm 14 is substantially straight and is typically rectangular in cross section, however, other shapes, such as elongated cylinders, can be used. In addition, the second arm 14 is typically about 0.75 to about 1 inch in thickness (or diameter), although other dimensions may be used. The second arm 14 is typically from about 35 inches to about 40 inches in length, and may be the same length as the first arm 12.

In the embodiment shown in FIG. 1, the second arm 14 includes at its distal end 14b a contacting portion 20 extending substantially perpendicularly from the first arm 12. The contacting portion 20 makes contact with the ruminant for which an estimate of body weight or ease of parturition is desired. The contacting portion 20 may be integrated with or attached to the second arm 14. Suitable attachment mechanisms include, for example, rivets, bolts, and adhesives.

The second arm 14 is made of a rigid material, such as maple or oak. Other variations of rigid material include other woods, metals, and hard plastics.

As shown in FIG. 1, the first arm 12 and the second arm 14 are pivotally mounted to each other at a location 22 between their proximal ends 12a and 14a and their distal ends 12b and 14b. In this particular embodiment, the first arm 12 and the second arm 14 are pivotally mounted to each other approximately half way between their proximal ends 12a and 14a and their distal ends 12b and 14b, although other locations of mounting may be used. Suitable devices for pivotally mounting the first arm 12 and the second arm 14 include, but are not limited to, a rivet or bolt, which allow movement and form a scissor-type action.

Referring to FIG. 1, the apparatus 10 further includes a scale device 16. In this particular embodiment, the scale device 16 is attached to the first arm 12, although the scale device may alternatively be attached to the second arm 14. The scale device 16 is attached to the first arm 12 at a position between the proximal end 12a and the distal end 12b. As shown in FIG. 1, in this particular embodiment, the scale device 16 is attached to the first arm 12 between the location 22 where the first arm 12 and second arm 14 are pivotally mounted to each other and the proximal end 12a, near the proximal end 12a. In accordance with this embodiment of the present invention, the scale device 16 is pivotally attached to the first arm 12 with, for example, a rivet or bolt, although other attachment mechanisms may be used. When rotated to a suitable position, the scale device 16 overlaps the second arm 14 at position 24. The scale device 16 includes markings which can be etched into or painted, printed, or written onto the scale device. The scale device 16 markings may be pounds, kilograms, centimeters, inches, combinations thereof, or any other suitable measurement.

The scale device 16 is made of a rigid material, such as a hardwood. Other variations of rigid material include metals and hard plastics.

Although not shown in FIG. 1, a guide or scale reader may be attached to the second arm 14, such that the scale device 16 slides through the guide and the guide forms a reference point to read the scale device 16. When the guide is present, the scale device 16 is of appropriate dimensions to fit within the guide.

In addition, the proximal ends 12a and 14a of the first arm 12 and the second arm 14, respectively, may also include a handle. The handle may be integrated with or attached to the proximal ends 12a and 14a. Suitable attachment mechanisms include, for example, rivets, bolts, and adhesives.

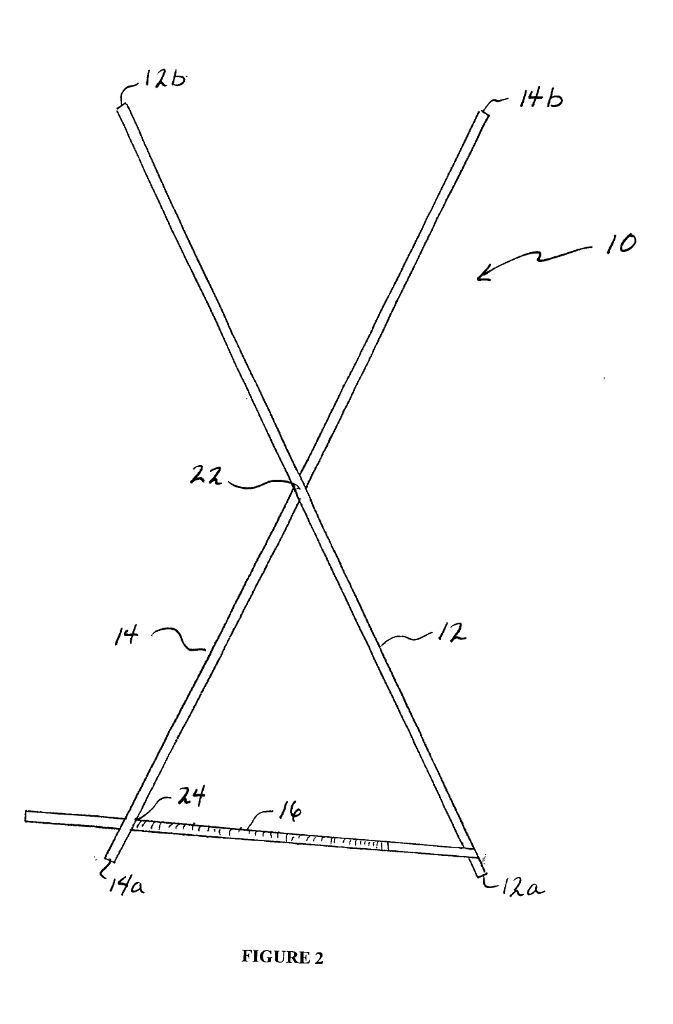

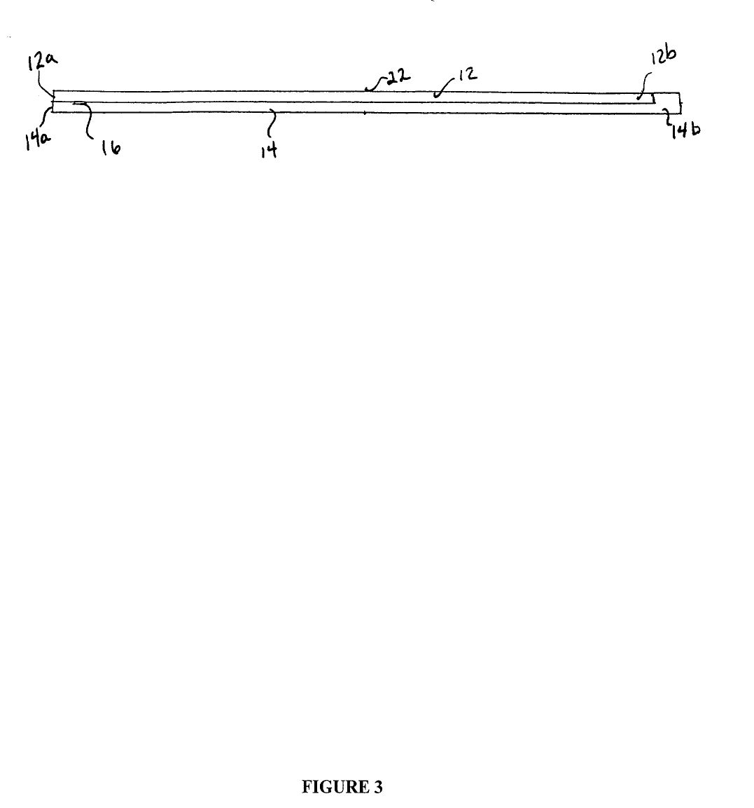

An apparatus 10 in accordance with a second embodiment of the present invention is shown in FIGS. 2 and 3. In this embodiment, the apparatus 10 is identical to the above-described apparatus except that it does not include contacting portions 18 and 20 at the distal ends 12b and 14b of the first arm 12 and the second arm 14, respectively. Thus, in accordance with this second embodiment of the present invention, the distal ends 12b and 14b of the first arm 12 and the second arm 14, respectively, are used to contact a ruminant.

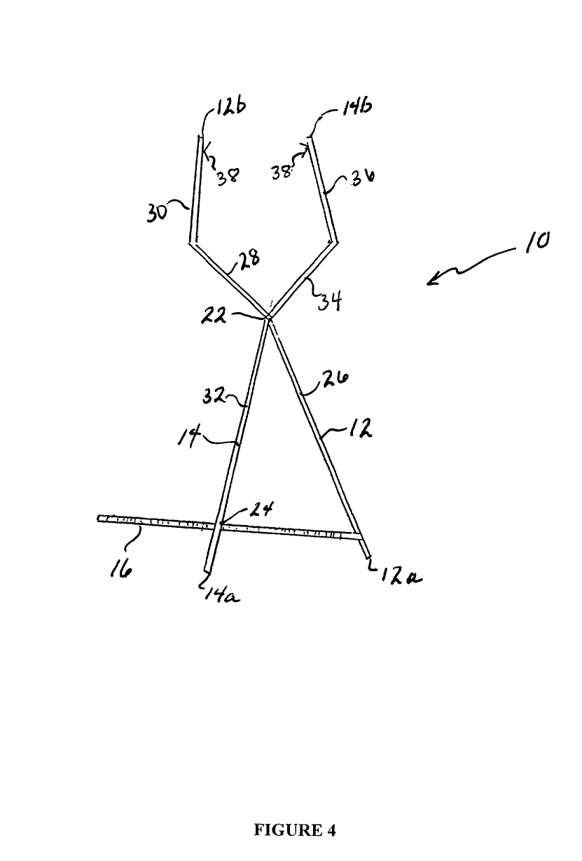

An apparatus 10 in accordance with a third embodiment of the present invention is shown in FIG. 4. The apparatus 10 in accordance with this embodiment of the invention is identical to the previous embodiments, except as described below.

Referring to FIG. 4, in this particular example the first arm 12 and the second arm 14 are configured to include three sections: a proximal section, an intermediate section, and a distal section. The proximal section (26 and 32, respectively) of the first and second arms extends from the proximal ends 12a and 14a, respectively, to the point 22 where the first arm 12 and the second arm 14 are attached. The portion of the first arm 12 and the second arm 14 between the pivot point 22 and their distal ends 12b and 14b, respectively, is configured in two sections (i.e., the intermediate section and the distal section) to form the two identical sides of an isosceles triangle. In particular, one end of the proximal section (26 and 32, respectively) is attached to a first end of the intermediate section (28 and 34, respectively) at an angle of from about 155° to about 165°, and the second end of the intermediate section (28 and 34, respectively) is attached to a first end of the distal section (30 and 36, respectively) at an angle of from about 135° to about 145°, although other dimensions may be used. The length of the first section (26 and 32, respectively) may be the same as or different than the length of the second (28 and 34, respectively) and third (30 and 36, respectively) sections. This configuration allows the device to be more easily placed on the desired location of a ruminant in order to estimate body weight or ease of parturition. In particular, this configuration allows the length of the first arm 12 and the second arm 14 to be shortened (while keeping the user at a safe distance), thus improving ease of use.

In addition, the distal ends 12a and 14a of the first arm 12 and the second arm 14, respectively, include convex devices 38 to guide the user to the correct anatomical location (contact point) for measurement, although other shapes may be used. The convex devices 38 are typically about 2 inches in diameter, although other dimensions may be used. The convex devices 38 may be integrated with or attached to the first and second arms. Suitable attachment mechanisms include, for example, rivets, bolts, and adhesives.

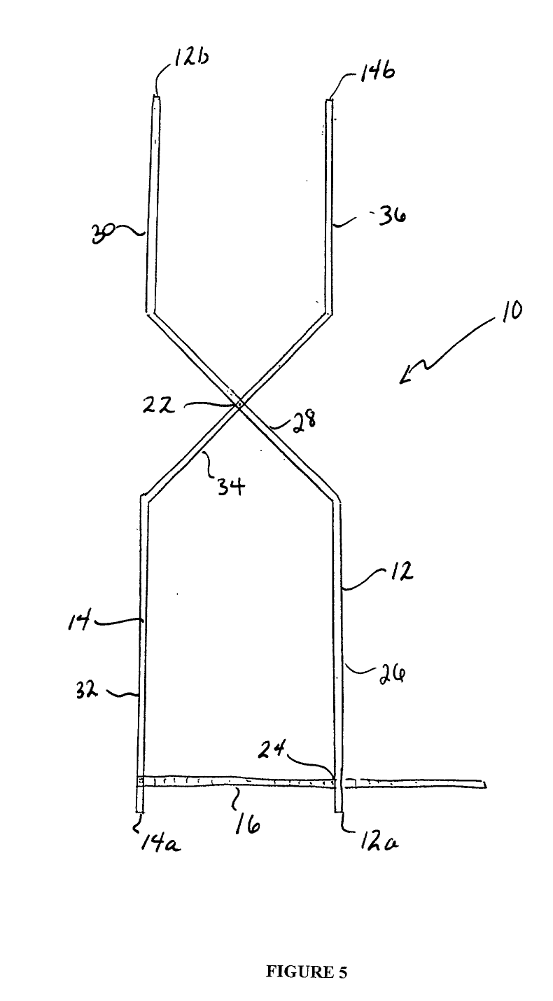

An apparatus 10 in accordance with a fourth embodiment of the present invention is shown in FIG. 5. The apparatus 10 in accordance with this embodiment of the invention is identical to the previous embodiments, except as described below.

Referring to FIG. 5, the scale device 16 is attached to the second arm 14 and overlaps the first arm 12. In addition, the first arm 12 and the second arm 14 are configured as “stretched out Z” shaped arm pieces, so that in the position shown in FIG. 5 (i.e., where the proximal sections of the first and second arms are held parallel), the proximal (12a and 14a) and distal (12b and 14b) ends of the first and second arms remain linearly aligned. In particular, in the embodiment shown in FIG. 5, the first arm 12 and second arm 14 comprise three sections: a proximal section, an intermediate section, and a distal section. One end of the proximal section (26 and 32, respectively) is attached to a first end of the intermediate section (28 and 34, respectively) at an angle of from about 150° to about 160°. The intermediate section 28 of the first arm 12 and the intermediate section 34 of the second arm 14 intersect at pivot point 22. The second end of the intermediate section (28 and 34, respectively) is attached to a first end of the distal section (30 and 36, respectively) at an angle of from about 150° to about 160°, wherein the angle between the proximal and intermediate sections is identical to the angle between the intermediate and distal sections. However, other dimensions may be used. The length of the three sections of the first arm 12 and the second arm 14 may be the same or different. This configuration allows the device to be easily placed on the desired location of the ruminant, and allows the length of the first arm 12 and the second arm 14 to be significantly shortened, while keeping the user at a safe distance.

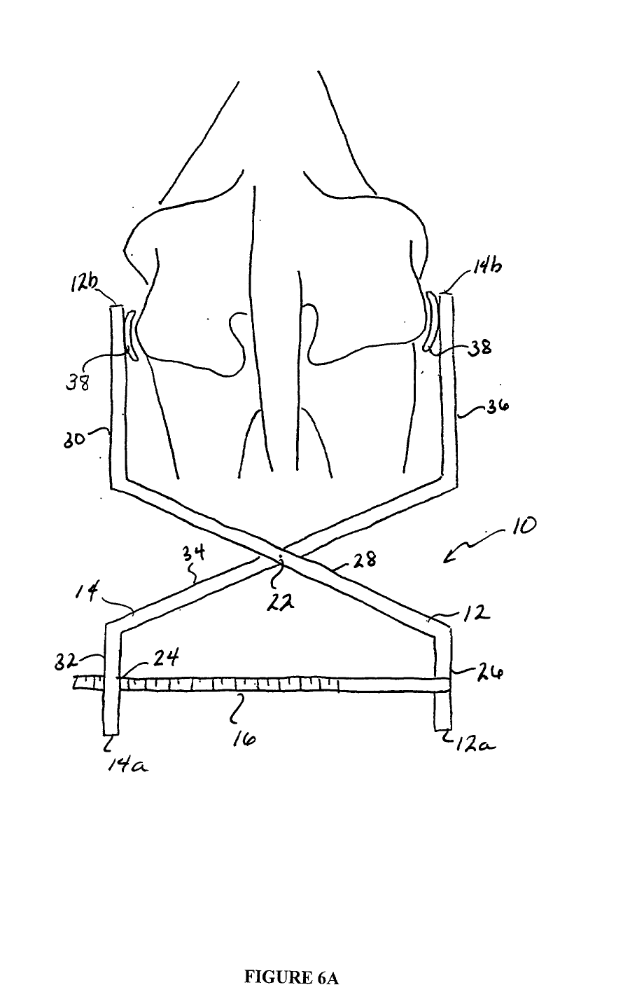

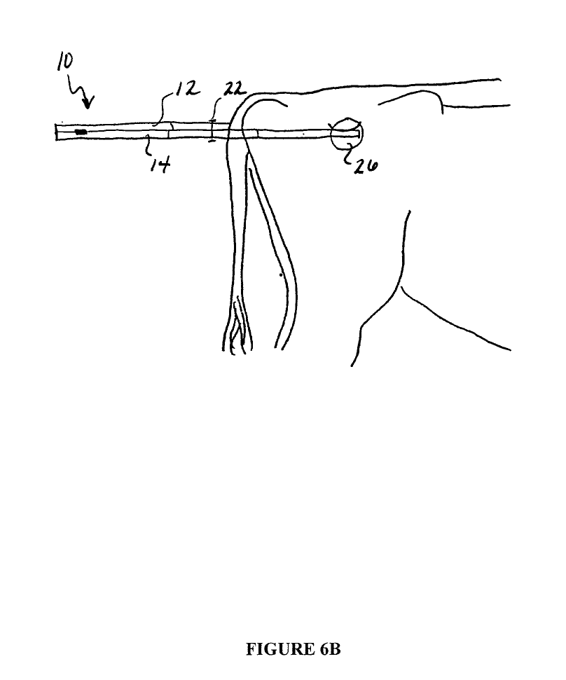

The apparatus 10 of the present invention is made providing a first arm 12, a second arm 14, and a scale device 16. The first arm 12 is pivotally attached to the second arm 14 so that the first and second arms function in a scissor-like manner. The first arm 12 is attached to the second arm 14 using a securing device, e.g. a rivet or bolt. Contacting portions 18 and 20, such as those shown in FIG. 1, or convex devices 38, such as those shown in FIGS. 6A and B, can be optionally integrated with or attached to distal portions 12b and 14b of the first and second arms. In addition, handles are optionally integrated with or attached to the proximal ends 12a and 14a of the first and second arms. Before or after the first and second arms are attached, the scale device 16 is pivotally attached near the proximal end of one of the first arm 12 or the second arm 14, so that it overlaps the other of the first arm 12 or the second arm 14.

The use of the apparatus 10 of the present invention will now be described in detail. In use, the user selects an apparatus 10 of appropriate size, based on the size of the ruminant being measured. Typically, the first arm 12, second arm 14, and scale device 16 will be pre-assembled into the apparatus 10. The user then places the distal ends 12b and 14b of the first and second arms, respectively, against the first and second sides of the caudal portion of the ruminant behind the tuber coxi, as shown in FIGS. 6A and B. For most accurate measurement, the user places the distal ends of the first and second arms against the greater trochanters on the left and right femurs of the ruminant. If present, the contacting portions 18 and 20 or convex devices 38 on the first and second arms, respectively, are placed against the greater trochanters on the left and right femurs of the ruminant. Referring to FIG. 6A, the scale device 16 is then positioned so that it overlaps the arm to which it is not attached, in this particular embodiment, the second arm 14. If a guide is present, the scale device 16 is positioned so that it slides through the guide and the guide forms a reference point to read the scale device 16. The scale device 16 is then read by the user. The scale device 16 may provide a measurement of width in appropriate units, or the scale device 16 may provide a converted measurement of body weight or ease of parturition. If the scale device 16 provides a measurement of width, the user then converts the width measurement to an estimate of body weight or ease of parturition.

The present invention also relates to a method for estimating body weight of a ruminant. As used herein, a ruminant is a multi-stomached animal, such as a bovine animal. Suitable bovine animals include cows, heifers, sheep, goats, calves, deer, buffalo, bison, elk, llamas, and alpacas. The ruminant includes a cranial or head portion, having first and second sides, and a caudal or tail portion, having first and second sides. Hip joints are located between the cranial and caudal portions.

In accordance with the method of the present invention, the distance between the hip joints of the ruminant is measured, as shown in FIGS. 6A and B. In this particular embodiment, the distance between greater trochanters of the left and right femurs of the ruminant is measured (the “pelvic width”). Distances of from about 5 cm to about 60 cm, typically, from about 20 cm to about 45 cm can be measured.

The measured distance is then converted to an estimated body weight of the ruminant. Suitable techniques for converting the measured distance to an estimated body weight include reading the measured distance and reading a weight assigned to the measured distance to estimate the body weight. Such body weights may be provided in a table or on a scale device. Body weights of from about 2 kg to about 750 kg, typically, from about 50 kg to about 355 kg can be estimated using the method of the present invention.

In accordance with the present invention, the pelvic width is a significant predictor of body weight. In particular, at least about 93% of the variation in body weight can be explained by pelvic width.

The present invention also relates to a method for estimating ease of parturition of a ruminant. This method of the present invention includes measuring a distance between hip joints of a ruminant and converting the measured distance to an estimated ease of parturition. As shown in FIGS. 6A and B, the step of measuring includes measuring the distance between the greater trochanters on the left and right femurs of the ruminant. In this particular embodiment, the step of converting includes reading the measured distance and reading an ease of parturition value assigned to the measured distance to estimate the ease of parturition. Such ease of parturition values may be provided in a table or on a scale device.

Another aspect of the present invention relates to a method for estimating a body weight or ease of parturition of a ruminant with a skeletal structure including a cranial portion having first and second sides, a caudal portion having first and second sides, and hip joints located between the cranial and caudal portions. This method includes measuring a distance behind tuber coxi of a ruminant (i.e., between the tuber coxi and the tail of the ruminant) and between first and second sides of a caudal portion of the ruminant and converting the measured distance to an estimated body weight or ease of parturition of the ruminant.

### EXAMPLES

### Example 1

### Measurement of Pelvic Width to Estimate Body Weight

The distance between greater trochanters on the left and right femurs (i.e. pelvic width) was measured on 240 Holstein heifers. Pelvic widths were measured to the nearest 0.5 cm. Body weights of the heifers were recorded with electronic scales at the same time pelvic widths were measured. Body weights of the 240 heifers ranged from about 55 kg to about 355 kg. Pelvic widths ranged from about 21.0 cm to about 43.5 cm.

A linear model, least square means analysis was conducted on the obtained data, with the results shown in Tables 1-5, below.

TABLE 1
Summary of Fit

| | |
|---|---|
| RSquare | 0.927661 |
| RSquare Adj. | 0.927358 |
| Root Mean Square Error | 19.471 |
| Mean of Response | 189.6949 |
| Observations (or Sum Wgts) | 240 |

TABLE 2
Lack of Fit

| Source | DF | Sum of Squares | Mean Square | F Ratio |
|---|---|---|---|---|
| Lack of Fit | 41 | 21044.406 | 513.276 | 1.4615 |
| Pure Error | 197 | 69186.100 | 351.198 | Prob > F |
| Total Error | 238 | 90230.507 | | 0.0469 |
| | | | | Max RSq |
| | | | | 0.9445 |

TABLE 3
Parameter Estimates

| Term | Estimate | Std Error | t Ratio | Prob > [t] |
|---|---|---|---|---|
| Intercept | −271.437 | 8.441025 | −32.16 | <0.0001 |
| Hip Width | 13.751876 | 0.248922 | 55.25 | <0.0001 |

TABLE 4
Effect Test

| Source | Nparm | DF | Sum of Squares | F Ratio | Prob > F |
|---|---|---|---|---|---|
| Hip width | 1 | 1 | 1157106.3 | 3052.086 | <0.0001 |

TABLE 5
Analysis of Variance

| Source | DF | Sum of Squares | Mean Square | F Ratio |
|---|---|---|---|---|
| Model | 1 | 1157106.3 | 1157106 | 3052.086 |
| Error | 238 | 90230.5 | 379 | Prob > F |
| C Total | 239 | 1247336.8 | | <0.0001 |

As shown in the above tables, the pelvic width measurement was a significant predictor of body weight (P<0.0001). In particular, the correlation between pelvic width and body weight was about 93% (R²=0.927). The pelvic width and body weight had a linear relationship within the weight range represented by this data set.

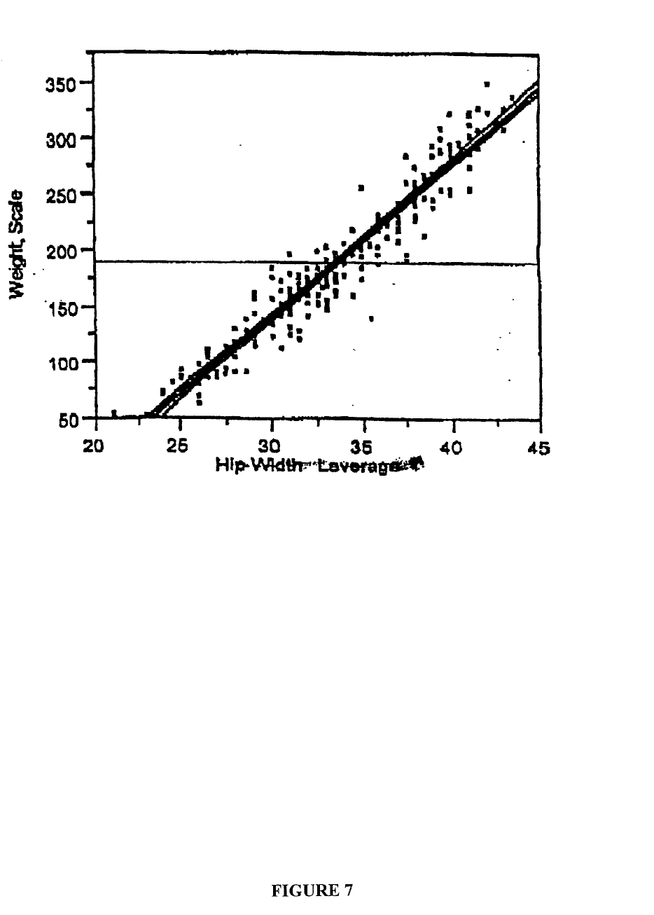

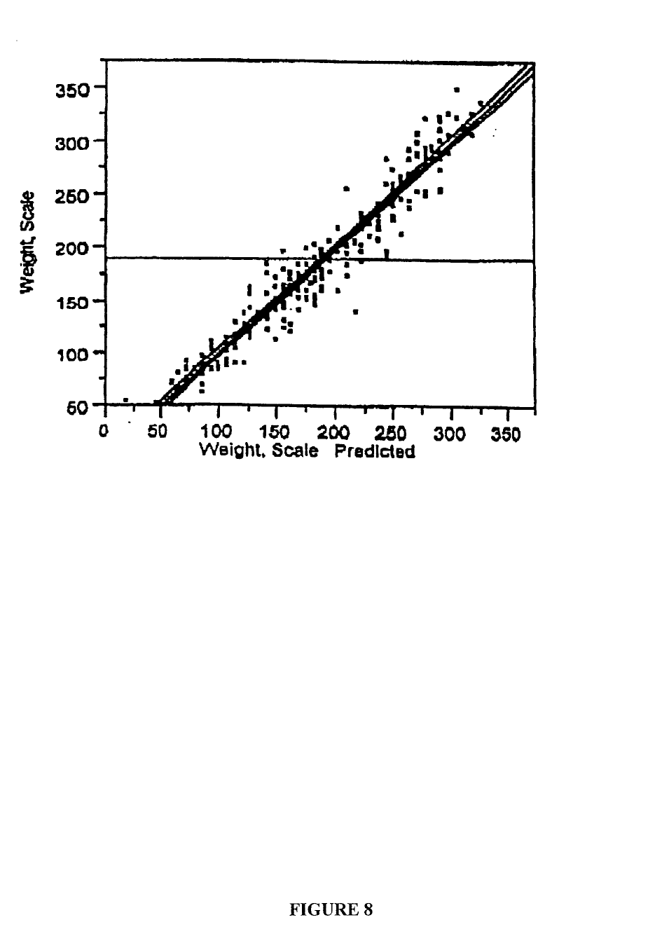

A leverage plot for the effect of pelvic width of Holstein heifers (FIG. 7) and a whole model leverage plot (FIG. 8) were produced based on the above analysis. These plots further indicated that pelvic width could be used to predict body weight, since the confidence curves in the plots crossed the horizontal line.

A quadratic term was added to the model and the correlation between hip width and body weight increased only slightly (R²=0.934), as shown in Tables 6-8, below (scale weight=−28.383−1.12711 hip width+0.22252 hip width²).

TABLE 6
Summary of Fit

| | |
|---|---|
| RSquare | 0.934399 |
| RSquare Adj. | 0.933845 |
| Root Mean Square Error | 18.58116 |
| Mean of Response | 189.6949 |
| Observations (or Sum Wgts) | 240 |

TABLE 7
Analysis of Variance

| Source | DF | Sum of Squares | Mean Square | F Ratio |
|---|---|---|---|---|
| Model | 2 | 1165510.3 | 582755 | 1687.876 |
| Error | 237 | 81826.5 | 345 | Prob > F |
| C Total | 239 | 1247336.8 | | <0.0001 |

TABLE 8
Parameter Estimates

| Term | Estimate | Std Error | t Ratio | Prob > [t] | Lower 95% | Upper 95% |
|---|---|---|---|---|---|---|
| Intercept | −28.38331 | 49.91851 | −0.57 | −0.5702 | −126.7252 | 69.958584 |
| Hip Width | −1.127112 | 3.025146 | −0.37 | 0.7098 | −7.086799 | 4.8325737 |
| Hip Width² | 0.2225158 | 0.045101 | 4.93 | <0.0001 | 0.1336637 | 0.3113679 |

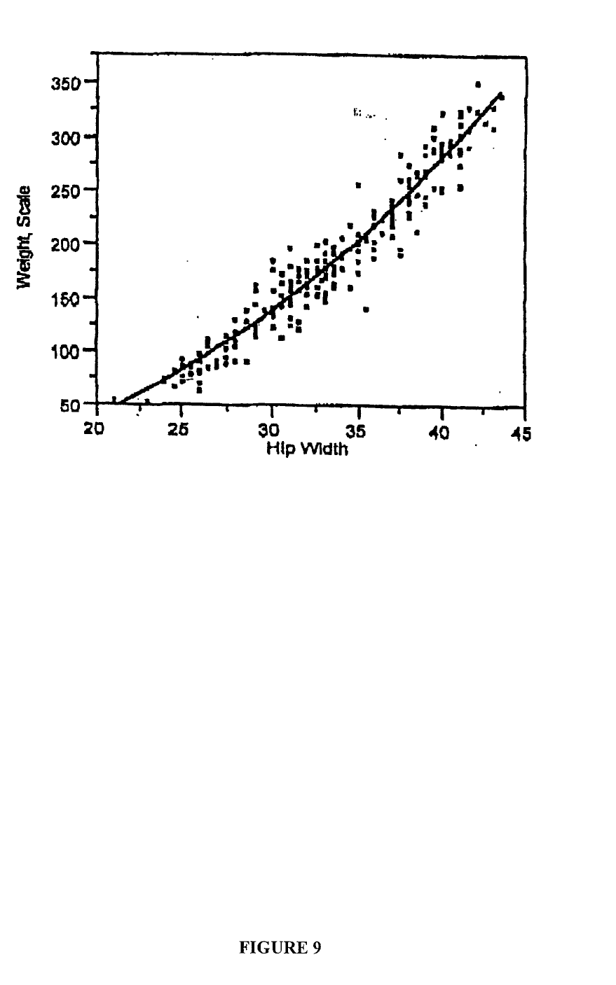

The resulting graph showing hip width versus scale width is shown in FIG. 9.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method for estimating a body weight of a ruminant with a skeletal structure including a cranial portion, a caudal portion, and hip joints located between the cranial and caudal portions comprising:
measuring a distance between hip joints of a ruminant; and
converting the measured distance to an estimated body weight of the ruminant.

2. The method according to claim 1 wherein the step of measuring comprises measuring the distance between greater trochanters on left and right femurs of the ruminant.

3. The method according to claim 1 wherein the step of converting comprises:
reading the measured distance; and
reading a weight assigned to the measured distance to estimate the body weight.

4. The method according to claim 1 wherein the ruminant is a bovine animal.

5. The method according to claim 4 wherein the bovine animal is selected from the group consisting of cows, heifers, sheep, goats, calves, deer, buffalo, bison, elk, llamas, and alpacas.

6. A method for estimating ease of parturition of a ruminant with a skeletal structure including a cranial portion, a caudal portion, and hip joints located between the cranial and caudal portions comprising:
measuring a distance between hip joints of a ruminant; and
converting the measured distance to an estimated ease of parturition value of the ruminant.

7. The method according to claim 6 wherein the step of measuring comprises measuring the distance between greater trochanters on left and right femurs of the ruminant.

8. The method according to claim 6 wherein the step of converting comprises:
reading the measured distance; and
reading an ease of parturition value assigned to the measured distance to estimate the ease of parturition.

9. The method according to claim 6 wherein the ruminant is a bovine animal.

10. The method according to claim 9 wherein the bovine animal is selected from the group consisting of cows, heifers, sheep, goats, calves, deer, buffalo, bison, elk, llamas, and alpacas.

11. A method for estimating a body weight or ease of parturition of a ruminant with a skeletal structure including a cranial portion having first and second sides, a caudal portion having first and second sides, and hip joints located between the cranial and caudal portions comprising:
measuring a distance behind tuber coxi of a ruminant and between first and second sides of a caudal portion of the ruminant; and
converting the measured distance to an estimated body weight or ease of parturition of the ruminant.

12. The method according to claim 11 wherein the step of measuring comprises measuring the distance between greater trochanters on left and right femurs of the ruminant.

13. The method according to claim 11 wherein the step of converting comprises:
reading the measured distance; and
reading a body weight value or an ease of parturition value assigned to the measured distance to estimate the body weight or ease of parturition.

14. The method according to claim 11 wherein the ruminant is a bovine animal.

15. The method according to claim 14 wherein the bovine animal is selected from the group consisting of cows, heifers, sheep, goats, calves, deer, buffalo, bison, elk, llamas, and alpacas.

16. An apparatus for estimating a body weight or ease of parturition of a ruminant comprising:
a first arm having proximal and distal ends;
a second arm having proximal and distal ends, wherein the first and second arms are pivotally mounted to each other between the proximal and distal ends of the first and second arms; and
a scale device connected to one of the first and second arms between the proximal and distal ends and overlapping the other one of the first arm and the second arm between the proximal and distal ends.

17. The apparatus according to claim 16 wherein the scale device is connected to the one of the first and second arms at a location between where the first and second arms are pivotally mounted to each other and the proximal end of the one of the first and second arms.

18. The apparatus according to claim 16 further comprising a handle at the proximal end of the first arm and the second arm.

19. The apparatus according to claim 16 further comprising a contacting device connected to the distal end of the first arm and the second arm.

20. The apparatus according to claim 19 wherein the contacting device is a convex device.

21. The apparatus according to claim 16 wherein the first arm and the second arm are substantially straight.

22. The apparatus according to claim 16 wherein the first arm and the second arm comprise:
a proximal section;
an intermediate section; and
a distal section, wherein the proximal section is adjacent and in contact with the intermediate section and wherein the intermediate section is adjacent and in contact with the distal section.

23. The apparatus according to claim 22 wherein the proximal section and the intermediate section are connected at an angle of from about 155° to about 165° and the intermediate section and the distal section are connected at an angle of from about 135° to about 145°.

24. The apparatus according to claim 22 wherein the proximal section and the intermediate section are connected at an angle of from about 150° to about 160° and the intermediate section and the distal section are connected at an angle of from about 150° to about 160°.
